(19) United States
(12) Patent Application Publication
(10) Pub. No.: US 2021/0313055 A1
(43) Pub. Date: Oct. 7, 2021

(54) MEDICAL CARE SUPPORTING SYSTEM AND MEDICAL CARE SUPPORTING METHOD

(71) Applicant: OLYMPUS CORPORATION, Tokyo (JP)

(72) Inventor: Yoshiko IKEDA, Tokyo (JP)

(73) Assignee: OLYMPUS CORPORATION, Tokyo (JP)

(21) Appl. No.: 17/351,361

(22) Filed: Jun. 18, 2021

Related U.S. Application Data
(63) Continuation of application No. PCT/JP2018/047293, filed on Dec. 21, 2018.

(51) Int. Cl. G16H 40/20; G16H 40/40

(52) U.S. Cl. CPC G16H 40/20; G16H 40/40

(57) ABSTRACT

An endoscope management unit 32 manages whether or not an endoscope is in a cleaned state. A staff management unit 34 manages whether or not a medical staff member is in a state suitable for being assigned to work of handling an endoscope in a cleaned state. A staff identification unit 36 identifies a staff member who performs work related to the endoscope based on the state of the endoscope managed by the endoscope management unit 32 and on the state of the medical staff member managed by the staff management unit 34. A notification processing unit 42 notifies the identified medical staff member of the details of the work to be performed for the endoscope.

### CROSS-REFERENCE TO RELATED APPLICATIONS

This application is based upon and claims the benefit of priority from International Application No. PCT/JP2018/047293, filed on Dec. 21, 2018, the entire contents of which are incorporated herein by reference.

### BACKGROUND

### 1. Field of the Disclosure

The present disclosure relates to a technology for supporting work of medical staff members.

### 2. Description of the Related Art

Japanese Patent Application Publication No. 2008-117382 provides a medical service support system that manages the usage status of medical instruments for which a work order is defined. This medical work support system reads out the usage status of a medical instrument before using the medical instrument for the work and determines whether or not the use of the medical instrument for the work follows the work order.

After being used for an examination, an endoscope is returned to a clean state after undergoing bedside cleaning in an examination room, water leakage inspection in a cleaning room, manual cleaning (preliminary cleaning), and mechanical cleaning (main cleaning). Upon removing an endoscope on which the main cleaning has been performed from a cleaning tank of a cleaning device, a medical staff member carries the endoscope to an examination room for the next examination or stores the endoscope in a storage. The hands of the medical staff member who handles this endoscope on which the main cleaning has been performed need to be clean. Therefore, if the hands are dirty, the hands must be washed before removing the endoscope from the cleaning tank, and this requires a predetermined period of time.

### SUMMARY

The present discloser considers that it is effective to notify a medical staff member of the details of work to be performed in order to improve work efficiency. A purpose of the present disclosure is to provide a technology for improving work efficiency of endoscopic examinations by appropriately selecting a medical staff member to be notified of the details of the work.

A medical care supporting system according to one embodiment of the present disclosure includes: an endoscope management unit that manages whether or not an endoscope is in a cleaned state; a staff management unit that manages whether or not a medical staff member is in a state suitable for being assigned to work of handling an endoscope in a cleaned state; a staff identification unit that identifies a staff member who performs work related to the endoscope based on the state of the endoscope managed by the endoscope management unit and on the state of the medical staff member managed by the staff management unit; and a notification processing unit that notifies the identified medical staff member of the details of the work to be performed for the endoscope.

Optional combinations of the aforementioned constituting elements and implementations of the present disclosure in the form of methods, apparatuses, systems, recording mediums, and computer programs may also be practiced as additional modes of the present disclosure.

### BRIEF DESCRIPTION OF THE DRAWINGS

Embodiments will now be described, by way of example only, with reference to the accompanying drawings that are meant to be exemplary, not limiting, and wherein like elements are numbered alike in several figures, in which:

FIG. 1 is a diagram showing the configuration of a medical care supporting system according to an embodiment;

FIG. 2 is a diagram showing functional blocks of a management device;

FIG. 3 is a diagram showing a status transition of an endoscope;

FIG. 4 is a diagram showing an example of an RFID reader table;

FIG. 5 is a diagram showing an example of a staff state table;

FIG. 6 is a diagram showing an example of a state management table of a staff; and

FIG. 7 is a diagram showing a flowchart of a staff identification process.

### DETAILED DESCRIPTION

The disclosure will now be described by reference to the preferred embodiments. This does not intend to limit the scope of the present disclosure, but to exemplify the disclosure.

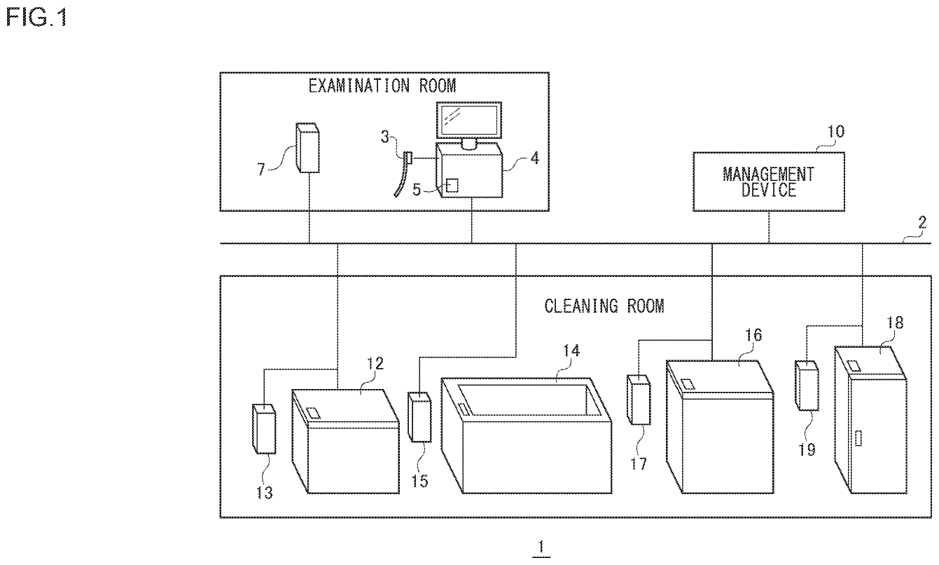

FIG. 1 shows the configuration of a medical care supporting system 1 according to an embodiment. Identification information (scope ID) is provided to an endoscope 3 in the medical care supporting system 1. Medical staff members such as nurses and technicians have identification information (staff IDs) for identifying themselves and perform work processes related to the endoscope 3 in accordance with an endoscopic examination work flow. A management device 10 manages the state of the endoscope 3 by using the scope ID and manages the state of the staff members by using the staff IDs. Via a network 2 such as a local area network (LAN), the management device 10 is communicably connected to an observation device 4 provided in an examination room and to a water leakage inspection device 12, a sink 14, a cleaning device 16, and a storage 18 provided in a cleaning room. The storage 18 may be placed in a storage room different from the cleaning room.

An ID reading device that reads a scope ID and/or a staff ID and transmits the scope ID and/or the staff ID to the management device 10 is provided at a place where a medical staff member performs each work. More specifically, an ID reading device 5 and an ID reading device 7 in the examination room, an ID reading device 13 provided near the water leakage inspection device 12, an ID reading device 15 provided near the sink 14, an ID reading device 17 provided near the cleaning device 16, and an ID reading device 19 provided near the storage 18 have a function of reading a scope ID and/or a staff ID and a function of transmitting the scope ID and/or the staff ID. In the examination room, the ID reading device 5 is used to read the scope ID of the endoscope 3 before the start of the examination, and the ID reading device 7 is used to read the scope ID of the endoscope 3 before the start of bedside cleaning. The ID reading device 5 and the ID reading device 7 may be commonalized. FIG. 1 shows one observation device 4, a water leakage inspection device 12, a sink 14, a cleaning device 16, and a storage 18. However, there may be a plurality of each of these items. When there are multiple observation devices 4, water leakage inspection devices 12, sinks 14, cleaning devices 16, and storages 18, ID reading devices may be provided for each of these items.

The medical staff member (also simply called “staff member”) may hold a portable terminal device and read the scope ID and staff ID using the terminal device at any point of time before the start of the work, during the work, or at the end of the work and transmit the scope ID and the staff ID to the management device 10 along with information specifying the work type. In any case, in the medical care supporting system 1, a mechanism for transmitting the scope ID and/or the staff ID to the management device 10 when the work is performed by the staff member is preferably prepared.

The management device 10 has a function of managing the state of an endoscope 3 and the state of staff members in a medical facility, identifying a staff member who performs the work related to the endoscope 3, and notifying the staff member. One requirement is that the staff member to be identified is not occupied with any work (is available) at that time. When the management device 10 identifies a staff member who performs the work, the management device 10 may notify the staff member of a work type to be performed and specific details of the work process in the work type. This allows the staff member to smoothly carry out the work related to the endoscope, and the efficiency of the endoscopic examination can thus be improved.

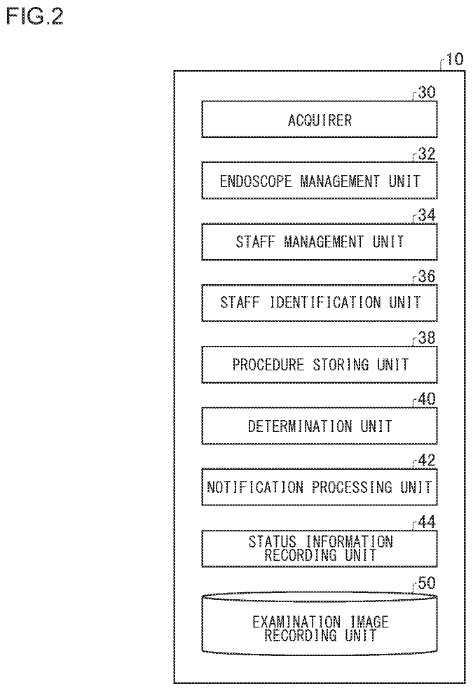

FIG. 2 illustrates functional blocks of the management device 10. The management device 10 includes an acquirer 30, an endoscope management unit 32, a staff management unit 34, a staff identification unit 36, a procedure storing unit 38, a determination unit 40, a notification processing unit 42, a status information recording unit 44, and an examination image recording unit 50. The status information recording unit 44 records the use and cleaning history of the endoscope 3 and the work history of a staff member.

The configuration thereof is implemented by hardware such as an arbitrary processor, a memory, auxiliary storage, or other LSIs and by software such as a program or the like loaded into the memory. The figure depicts functional blocks implemented by the cooperation of hardware and software. Thus, a person skilled in the art should appreciate that there are many ways of accomplishing these functional blocks in various forms in accordance with the components of hardware only, software only, or the combination of both.

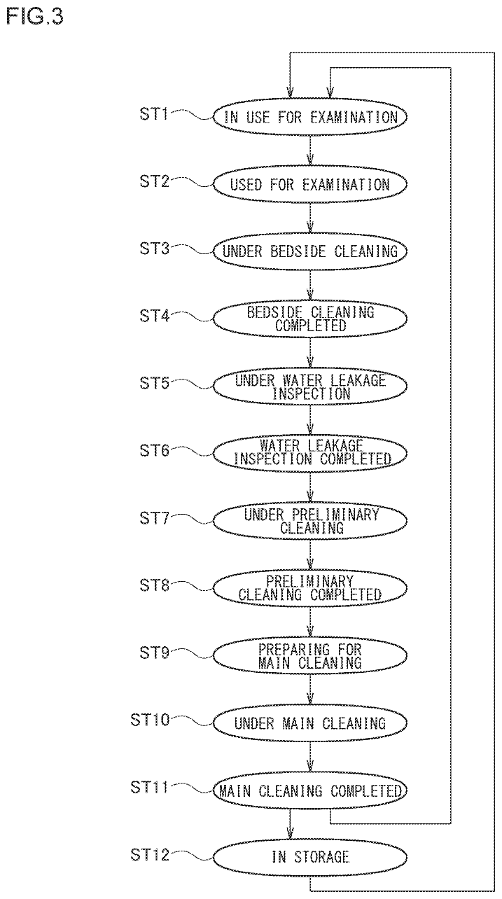

FIG. 3 shows the status transition of the endoscope 3 in the embodiment. The management device 10 manages the status of the endoscope 3 using statuses of “in use for examination” (ST1), “used for examination” (ST2), “under bedside cleaning” (ST3), “bedside cleaning completed” (ST4), “under water leakage inspection” (ST5), “water leakage inspection completed” (ST6), “under preliminary cleaning” (ST7), “preliminary cleaning completed” (ST8), “preparing for main cleaning” (ST9), “under main cleaning” (ST10), “main cleaning completed” (ST11), and “in storage” (ST12). In FIG. 3, the direction of an arrow indicates a typical transition direction of the endoscopic status.

The staff member has an ID card on which the staff ID is recorded, and the ID card may be brought into contact with or held over an ID reading device installed at the work place so that the staff ID is read by the ID reading device. Further, a tape on which the scope ID is printed is attached to the endoscope 3, and the staff member may bring the taped part into contact with the ID reading device or holding the taped part over the ID reading device so that the scope ID is read by the ID reading device. The scope ID may be attached to the endoscope 3 in a form other than tape (for example, in a waterproofed form), as long as the scope ID is readable by the ID reading device. The staff member may have an RFID tag on which the staff ID is recorded, and the endoscope 3 may have an RFID tag on which the scope ID is recorded. At this time, the staff member causes the ID reading device, which is an RFID reader, to read the staff ID and the scope ID.

In the following example, the staff ID and the scope ID are each recorded on an RFID tag, and each ID reading device in the medical care supporting system 1 is an RFID reader and realizes non-contact ID reading. Each ID reading device transmits the read staff ID and scope ID to the management device 10 along with the device ID of each ID reading device. The management device 10 stores the device ID of an ID reading device in association with the work type performed at the installation location of the ID reading device. Therefore, the management device 10 can manage the work type performed by the staff member regarding the endoscope 3 by acquiring the device ID of each ID reading device.

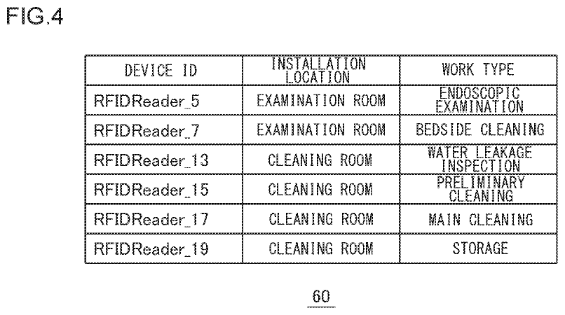

FIG. 4 shows an example of an RFID reader table 60 that describes the association between the device IDs of the ID reading devices and work types. The RFID reader table 60 records the installation locations of the ID reading devices and work types in association with the device IDs of the ID reading devices. The endoscope management unit 32 and the staff management unit 34 manage the state of the endoscope 3 and the state of the staff member with reference to the RFID reader table 60. The RFID reader table 60 may be stored in the status information recording unit 44.

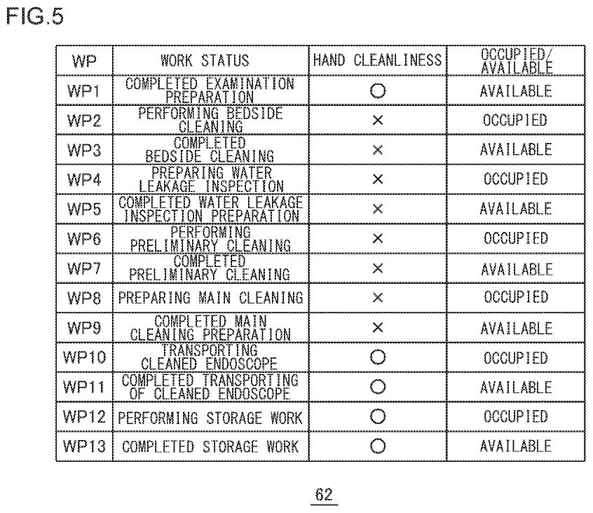

FIG. 5 shows a staff state table 62 for managing the state of staff members. The staff management unit 34 manages the work status of a staff member based on the work performed by the staff member for the endoscope. The staff management unit 34 manages the status of the work status of a staff member using statuses of “completed examination preparation” (WP1), “performing bedside cleaning” (WP2), “completed bedside cleaning” (WP3), “preparing water leakage inspection” (WP4), “completed water leakage inspection preparation” (WP5), “performing preliminary cleaning” (WP6), “completed preliminary cleaning” (WP7), “preparing main cleaning” (WP8), “completed main cleaning preparation” (WP9), “transporting cleaned endoscope” (WP10), “completed transporting of cleaned endoscope” (WP11), “performing storage work” (WP12), and “completed storage work” (WP13).

In the staff state table 62, each work status is associated with information indicating the cleanliness of the staff member's hands and information indicating whether the staff member is in a working state or in an available state. The information indicating the cleanliness of the hands may be set as a flag value. A state in which the hands are clean is defined as a state in which an endoscope 3 on which the main cleaning has been performed can be handled without the need for hand washing, and a state in which the hands are not clean is defined as a state in which hand washing is required in order to handle an endoscope 3 on which the main cleaning has been performed.

A “working” state indicates that the staff member is performing the work indicated by a work status. Therefore, when new work to be performed on the endoscope 3 is generated, the management device 10 cannot assign the work to a staff member who is in the state of “working”. On the other hand, an “available” state indicates that the staff member has completed the work indicated by a work status. Therefore, when new work to be performed on the endoscope 3 is generated, the management device 10 can assign the work to a staff member who is in the state of “available”.

The staff management unit 34 manages the state of a staff member with reference to the RFID reader table 60 and the staff state table 62. Described below is the operation of each structure in the medical care supporting system 1 when the status of the endoscope 3 transitions according to the transition diagram shown in FIG. 3. In this explanation, those who perform work related to the endoscope 3 are collectively referred to as “staff members” in all the work. However, the same staff member does not need to perform each work.

Before the start of the examination, the staff causes the staff ID and the scope ID of the endoscope 3 to be read by the ID reading device 5, and then connects the endoscope 3 to the observation device 4. The ID reading device 5 transmits the scope ID and the staff ID to the management device 10 along with the device ID (RFIDReader_5) of the ID reading device 5. When an examination start button provided on the observation device 4 is operated, the observation device 4 transmits information indicating the start of the examination to the management device 10, and the endoscope examination is started. During the examination, the observation device 4 displays image data acquired by the endoscope 3 on a monitor in real time, acquires an examination image at the time when a release switch of the endoscope 3 is pressed, and transmits the examination image to the management device 10. The transmission destination of the examination image may be a network storage or an image management system other than the management device 10. Further, the acquired examination image may be accumulated in the observation device 4 and read out to a USB memory or the like after the examination is completed. When an examination completion button provided on the observation device 4 is operated, the observation device 4 transmits information indicating the completion of the examination to the management device 10, and the endoscope examination is ended.

Before the start of the examination, when the ID reading device 5 transmits the read staff ID and scope ID along with the device ID (RFIDReader_5) to the management device 10, the acquirer 30 acquires the device ID (RFIDReader_5), the staff ID, and the scope ID. The acquirer 30 records the device ID (RFIDReader_5), the staff ID, and the scope ID in the status information recording unit 44 along with the acquisition time.

The endoscope management unit 32 identifies the work type based on the device ID (RFIDReader_5) recorded in the status information recording unit 44 in reference to the RFID reader table 60. The work type is “endoscopic examination”, and it is identified that an endoscope 3 corresponding to the scope ID is used for the endoscopic examination. When the acquirer 30 acquires the examination start information from the observation device 4, the endoscope management unit 32 sets the status of the endoscope 3 to “in use for examination” (ST1).

The staff management unit 34 identifies that the work type associated with the device ID (RFIDReader_5) is “endoscopic examination” in reference to the RFID reader table 60. When the acquirer 30 acquires the examination start information from the observation device 4, the staff management unit 34 sets the status of a staff member corresponding to the staff ID to “completed examination preparation” (WP1) in reference to the staff state table 62. The staff member who has been set to “completed examination preparation” (WP1) has clean hands and is in a state of being able to receive instructions for new work.

During the examination, when the observation device 4 transmits the examination image data to the management device 10, the acquirer 30 acquires the examination image data and records the examination image data in the examination image recording unit 50. After that, when the acquirer 30 acquires the examination completion information from the observation device 4, the acquisition time and the examination completion information are recorded in the status information recording unit 44 in association with the scope ID. This acquisition time corresponds to the examination completion time. At this time, the endoscope management unit 32 sets the status of the endoscope 3 to “used for examination” (ST2).

After the examination is completed, the staff member wipes the outer surface of the endoscope and performs a suction cleaning of a suction/forceps channel in the examination room. This cleaning process is also called bedside cleaning. As mentioned above, a staff member who performs the bedside cleaning may be different from a staff member who prepares the examination.

The staff member who performs the bedside cleaning causes the staff ID to be read by the ID reading device 7, and then causes the scope ID of the endoscope 3 to be read by the ID reading device 7. Before the start of the bedside cleaning, the ID reading device 7 transmits the read staff ID and scope ID to the management device 10 along with the device ID (RFIDReader_7). In the management device 10, the acquirer 30 acquires the device ID (RFIDReader_7), the staff ID, and the scope ID and records the device ID (RFIDReader_7), the staff ID, and the scope ID in the status information recording unit 44 along with the acquisition time. The acquisition time corresponds to the start time of the bedside cleaning.

The endoscope management unit 32 identifies that the work type associated with the device ID (RFIDReader_7) is “bedside cleaning” in reference to the RFID reader table 60. The endoscope management unit 32 sets the status of the endoscope 3 to “under bedside cleaning” (ST3).

The staff management unit 34 identifies that the work type associated with the device ID (RFIDReader_7) is “bedside cleaning” in reference to the RFID reader table 60.

The staff management unit 34 sets the status of the staff member to “performing bedside cleaning” (WP2) in reference to the staff state table 62. The staff member who has been set to “performing bedside cleaning” (WP2) has unclean hands and is in a state of being unable to receive instructions for new work since he/she is occupied with work.

The staff member starts the bedside cleaning after causing the staff ID and the scope ID to be read by the ID reading device 7. In the bedside cleaning, the staff member wipes the outer surface of the endoscope and performs suction cleaning of a suction/forceps channel. Upon completing the bedside cleaning, the staff member causes the staff ID and the scope ID to be read by the ID reading device 7. The ID reading device 7 transmits the read staff ID and scope ID to the management device 10 along with the device ID (RFIDReader_7). Upon acquiring the device ID, the staff ID, and the scope ID from the ID reading device 7 in the management device 10, the acquirer 30 records the device ID, the staff ID, and the scope ID in the status information recording unit 44 along with the acquisition time. This acquisition time corresponds to the end time of the bedside cleaning. When the status of the endoscope 3 before the acquisition is “under bedside cleaning” (ST3) and the same scope ID is newly transmitted from the ID reading device 7, the endoscope management unit 32 determines that the bedside cleaning is completed and sets the status of the endoscope 3 to “bedside cleaning completed” (ST4).

When the status of the staff member before the acquisition of the staff ID is “performing bedside cleaning” (WP2) and the same staff ID is newly transmitted from the ID reading device 7, the staff management unit 34 determines that the bedside cleaning is completed and sets the status of the staff member to “completed bedside cleaning” (WP3). The staff member who has been set to “completed bedside cleaning” (WP3) has unclean hands and is in a state of being able to receive instructions for new work.

When the bedside cleaning of the endoscope is completed, the staff member puts the endoscope 3 on which the bedside cleaning has been performed into a dedicated container and carries the container to the cleaning room. In the cleaning room, the carried-in endoscope 3 is subjected to each work of water leakage inspection, preliminary cleaning, and main cleaning, and is returned to a clean state that can be reused for an examination.

The cleaning room is provided with a water leakage inspection device 12 for inspecting the carried-in endoscope 3 for water leakage, a sink 14 for a staff member to perform manual cleaning (preliminary cleaning) on the endoscope 3 on which the water leakage inspection has been performed, a cleaning device 16 for performing automatic mechanical cleaning on the endoscope 3 on which the preliminary cleaning has been performed, and a storage 18 for storing the endoscope 3 on which the automatic mechanical cleaning (main cleaning) has been performed. The endoscope 3 on which the main cleaning has been performed is stored in the storage 18. Alternatively, the endoscope 3 may be directly transported to an examination room when the endoscope 3 is to be used for an examination immediately.

A staff member who performs the water leakage inspection causes the staff ID to be read by the ID reading device 13 and then causes the scope ID of the endoscope 3 to be read by the ID reading device 13 before the water leakage inspection. The ID reading device 13 transmits the read staff ID and scope ID to the management device 10 along with the device ID (RFIDReader_13). After causing the staff ID and the scope ID to be read by the ID reading device 13, the staff member sets the endoscope 3 on which the bedside cleaning has been performed in the water leakage inspection device 12 and operates an inspection start button to start the water leakage inspection. When the examination start button is operated, the water leakage inspection device 12 transmits the examination start information to the management device 10 along with the device ID (RFIDReader_13).

In the management device 10, the acquirer 30 acquires the device ID (RFIDReader_13), the staff ID, and the scope ID from the ID reading device 13 and records the device ID, the staff ID, and the scope ID in the status information recording unit 44. The staff management unit 34 identifies that the work type associated with the device ID (RFIDReader_13) is “water leakage inspection” in reference to the RFID reader table 60. The staff management unit 34 sets the status of the staff member to “preparing water leakage inspection” (WP4) in reference to the staff state table 62. The staff member who has been set to “preparing water leakage inspection” (WP4) has unclean hands and is in a state of being unable to receive instructions for new work since he/she is occupied with work.

Upon acquiring the inspection start information from the water leakage inspection device 12, the acquirer 30 records the acquisition time in the status information recording unit 44 in association with the device ID, the staff ID, and the scope ID acquired before the inspection start information. The acquisition time corresponds to the start time of the water leakage inspection. In reference to the RFID reader table 60, the endoscope management unit 32 identifies that the work type associated with the device ID (RFIDReader_13) is “water leakage inspection”, and sets the status of the endoscope 3 to “under water leakage inspection” (ST5).

When the acquisition time of the inspection start information of the water leakage inspection is recorded in the status information recording unit 44, the staff management unit 34 determines that the water leakage inspection preparation is completed and sets the status of the staff member to “completed water leakage inspection preparation” (WP5). The staff member who has been set to “completed water leakage inspection preparation” (WP5) has unclean hands and is in a state of being able to receive instructions for new work.

When the water leakage inspection is completed, the water leakage inspection device 12 automatically transmits the completion information of the water leakage inspection to the management device 10. When the acquirer 30 acquires the completion information of the water leakage inspection in the management device 10, the completion information of the water leakage inspection is recorded in the status information recording unit 44 along with the acquisition time. This acquisition time corresponds to the end time of the water leakage inspection. At this time, the endoscope management unit 32 sets the status of the endoscope 3 to “water leakage inspection completed” (ST6).

Before the start of the preliminary cleaning, a staff member for the preliminary cleaning first causes the ID reading device 15 installed next to the sink 14 to read the staff ID and then the scope ID of the endoscope 3 confirmed to have no water leakage. The ID reading device 15 transmits the read staff ID and scope ID to the management device 10 along with the device ID (RFIDReader_15). In the management device 10, the acquirer 30 acquires the device ID (RFIDReader_15), the staff ID, and the scope ID and records the device ID (RFIDReader_15), the staff ID, and the scope ID in the status information recording unit 44 along with the acquisition time. The acquisition time corresponds to the start time of the preliminary cleaning.

The endoscope management unit 32 identifies that the work type associated with the device ID (RFIDReader_15) is “preliminary cleaning” in reference to the RFID reader table 60. The endoscope management unit 32 sets the status of the endoscope 3 to “under preliminary cleaning” (ST7).

The staff management unit 34 identifies that the work type associated with the device ID (RFIDReader_15) is “preliminary cleaning” in reference to the RFID reader table 60. The staff management unit 34 sets the status of the staff member to “performing preliminary cleaning” (WP6) in reference to the staff state table 62. The staff member who has been set to “performing preliminary cleaning” (WP6) has unclean hands and is in a state of being unable to receive instructions for new work since he/she is occupied with work.

The staff member for the preliminary cleaning starts the preliminary cleaning of the endoscope 3 after causing the staff ID and the scope ID to be read by the ID reading device 15. The staff member removes an air supply/water supply button, a suction button, a forceps plug, and the like from the endoscope and cleans the outer surface of the endoscope with running water. The staff member then brushes the suction/forceps channel using a dedicated channel cleaning brush. Further, the cleaning staff member cleans the removed air supply/water supply button, suction button, and forceps plug using a brush.

Upon completing the preliminary cleaning, the staff member causes the staff ID and the scope ID to be read by the ID reading device 15. The ID reading device 15 transmits the read staff ID and scope ID to the management device 10 along with the device ID (RFIDReader_15). Upon acquiring the device ID, the staff ID, and the scope ID from the ID reading device 15 in the management device 10, the acquirer 30 records the device ID, the staff ID, and the scope ID in the status information recording unit 44 along with the acquisition time. This acquisition time corresponds to the end time of the preliminary cleaning. When the status of the endoscope 3 before the acquisition is “under preliminary cleaning” (ST7) and the same scope ID is newly transmitted from the ID reading device 15, the endoscope management unit 32 determines that the preliminary cleaning is completed and sets the status of the endoscope 3 to “preliminary cleaning completed” (ST8).

When the status of the staff member before the acquisition of the staff ID is “performing preliminary cleaning” (WP6) and the same staff ID is newly transmitted from the ID reading device 15, the staff management unit 34 determines that the preliminary cleaning is completed and sets the status of the staff member to “completed preliminary cleaning” (WP7). The staff member who has been set to “completed preliminary cleaning” (WP7) has unclean hands and is in a state of being able to receive instructions for new work.

Before the start of the main cleaning, a staff member for the main cleaning causes the ID reading device 17 arranged in the cleaning device 16 to read the staff ID and the scope ID in this order. The ID reading device 17 transmits the read staff ID and scope ID to the management device 10 along with the device ID (RFIDReader_17). After having the staff ID and the scope ID read by the ID reading device 17, the staff member sets the endoscope 3 on which the preliminary cleaning has been performed in the cleaning tank of the cleaning device 16, selects a cleaning program for the endoscope 3, and operates a cleaning start button so as to start machine cleaning. When the cleaning start button is operated, the cleaning device 16 transmits cleaning start information to the management device 10 along with the device ID (RFIDReader_17).

In the management device 10, the acquirer 30 acquires the device ID (RFIDReader_17), the staff ID, and the scope ID from the ID reading device 17 and records the device ID, the staff ID, and the scope ID in the status information recording unit 44. The endoscope management unit 32 identifies that the work type associated with the device ID (RFIDReader_17) is “main cleaning” in reference to the RFID reader table 60. The endoscope management unit 32 sets the status of the endoscope 3 to “preparing for main cleaning” (ST9).

The staff management unit 34 identifies that the work type associated with the device ID (RFIDReader_17) is “main cleaning” in reference to the RFID reader table 60. The staff management unit 34 sets the status of the staff member to “preparing main cleaning” (WP8) in reference to the staff state table 62. The staff member who has been set to “preparing main cleaning” (WP8) has unclean hands and is in a state of being unable to receive instructions for new work since he/she is occupied with work.

Upon acquiring the cleaning start information from the cleaning device 16, the acquirer 30 records the acquisition time in the status information recording unit 44 in association with the device ID, the staff ID, and the scope ID acquired before the inspection start information. The acquisition time corresponds to the start time of the main cleaning. In reference to the RFID reader table 60, the endoscope management unit 32 identifies that the work type associated with the device ID (RFIDReader_17) is “main cleaning”, and sets the status of the endoscope 3 to “under main cleaning” (ST10).

When the acquisition time of main cleaning start information is recorded in the status information recording unit 44, the staff management unit 34 determines that the main cleaning preparation is completed and sets the status of the staff member to “completed main cleaning preparation” (WP9). The staff member who has been set to “completed main cleaning preparation” (WP9) has unclean hands and is in a state of being able to receive instructions for new work.

When the main cleaning is completed, the cleaning device 16 automatically transmits cleaning completion information to the management device 10. When the acquirer 30 acquires the completion information of the main cleaning in the management device 10, the completion information of the main cleaning is recorded in the status information recording unit 44 in association with the scope ID recorded at the start of the main cleaning along with the acquisition time. The acquisition time corresponds to the completion time of the main cleaning. At this time, the endoscope management unit 32 sets the status of the endoscope 3 to “main cleaning completed” (ST11).

After the main cleaning is completed, the staff member transports the endoscope 3 on which the main cleaning has been performed to a predetermined location. The location to be transported to is the examination room or the storage 18, and the location to be transported to is instructed to the staff member by the management device 10. The staff member causes the ID reading device 17 arranged in the cleaning device 16 to read the staff ID. The ID reading device 17 transmits the read staff ID to the management device 10 along with the device ID (RFIDReader_17). After having the staff ID read by the ID reading device 17, the staff member opens the lid of the cleaning device 16 and takes out the cleaned endoscope 3 from the cleaning tank and transports the endoscope 3 to a predetermined location. In this example, it is assumed that the staff member is instructed to transport the endoscope to the storage 18 by the management device 10.

In the management device 10, the acquirer 30 acquires the device ID (RFIDReader_17) and the staff ID from the ID reading device 17 and records the device ID and the staff ID in the status information recording unit 44. The staff management unit 34 identifies that the work type associated with the device ID (RFIDReader_17) is “main cleaning” in reference to the RFID reader table 60. Since the cleaning of the endoscope in the cleaning device 16 has been completed, the staff management unit 34 sets the status of the staff member to “transporting cleaned endoscope” (WP10) in reference to the staff state table 62. The staff member who has been set to “transporting cleaned endoscope” (WP10) has clean hands and is in a state of being unable to receive instructions for new work since he/she is occupied with work.

When the staff member carries the endoscope on which the main cleaning has been performed to the storage 18, the staff member causes the ID reading device 19 arranged in the storage 18 to read the staff ID and the scope ID in this order. The ID reading device 19 transmits the read staff ID and scope ID to the management device 10 along with the device ID (RFIDReader_19). The acquirer 30 acquires the device ID (RFIDReader_19), the staff ID, and the scope ID from the ID reading device 19 and records the device ID, the staff ID, and the scope ID in the status information recording unit 44.

The staff management unit 34 identifies that the work type associated with the device ID (RFIDReader_19) is “storage” in reference to the RFID reader table 60. Since the current status of the staff member is “transporting cleaned endoscope” (WP10), the staff management unit 34 sets the status of the staff member to “completed transporting of cleaned endoscope” (WP11) in reference to the staff state table 62. The staff member who has been set to “completed transporting of cleaned endoscope” (WP11) has clean hands and is in a state of being able to receive instructions for new work.

Next, the staff management unit 34 sets the status of the staff member to “performing storage work” (WP12) in reference to the staff state table 62. In the embodiment, the status of the staff member is changed from “completed transporting of cleaned endoscope” (WP11) to “performing storage work” (WP12). Alternatively, the staff management unit 34 may change the status from “transporting cleaned endoscope” (WP10) directly to “performing storage work” (WP12).

The staff member opens the door of the storage 18, stores the endoscope in the storage 18, closes the door, and then causes the ID reading device 19 to read the staff ID. The ID reading device 19 transmits the read staff ID to the management device 10 along with the device ID (RFIDReader_19). Upon acquiring the device ID (RFIDReader_19) and the staff ID from the ID reading device 19, the acquirer 30 records the device ID (RFIDReader_19) and the staff ID in the status information recording unit 44 along with the acquisition time. At this time, the endoscope management unit 32 sets the status of the endoscope 3 to “in storage” (ST12). Further, the staff management unit 34 sets the status of the staff member to “completed storage work” (WP13).

The management device 10 manages the status of an endoscope 3 and the work status of a staff member as described above. In the status transition shown in FIG. 3, new work to be performed is generated when the endoscope 3 transitions to the status ST2, ST4, ST6, ST8, or ST11.

More specifically, an endoscope in the status ST2 needs to go through the bedside cleaning, an endoscope in the status ST4 needs to go through the water leakage inspection, an endoscope in the status ST6 needs to go through the preliminary cleaning, and an endoscope in the status ST8 needs to go through the main cleaning. An endoscope in the status ST11 needs to be transported to the storage or the examination room. Further, when an examination requires an endoscope in the status ST12, the endoscope needs to be taken out from the storage 18 and carried to the examination room.

Efficient endoscopic examinations can be realized by promptly carrying out work related to endoscopes by staff members. Therefore, the management device 10 manages the status of an endoscope 3 in real time, and when the endoscope 3 is in a status where new work is required, the management device 10 searches for a staff member capable of performing the work and gives instructions regarding the details of the work. For example, each staff member wears a voice output device such as a headset, and by sending work instructions by voice to staff members capable of performing the work, the management device 10 enables the staff member to immediately grasp the details of the work and respond.

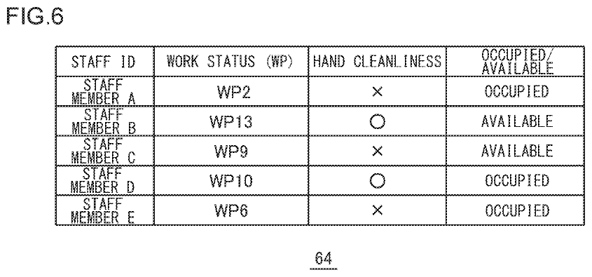

FIG. 6 shows an example of a state management table 64 of each staff member managed by the staff management unit 34. The state management table 64 may be created by the staff management unit 34 and stored in the status information recording unit 44. The staff management unit 34 may update the state management table 64 every time the acquirer 30 acquires the staff ID.

In the management device 10 according to the embodiment, the staff identification unit 36 identifies a staff member who performs work related to the endoscope based on the state of the endoscope managed by the endoscope management unit 32 and on the state of staff members managed by the staff identification unit 36. As to the timing for identifying the staff member, the identification may be carried out when the status of the endoscope transitions to any one of ST2, ST4, ST6, ST8, and ST11 as described above.

The endoscope management unit 32 manages the status of all endoscopes in the medical facility. When the statuses ST1 to ST12 of an endoscope are classified from the viewpoint of whether or not the endoscope is in a cleaned state, ST1 to ST10 represent a state where the endoscope is uncleaned, and ST11 to ST12 represent a state where the endoscope is cleaned. Therefore, the endoscope management unit 32 manages whether or not the endoscope is in a cleaned state by managing the status of the endoscope based on the work performed for the endoscope.

Since an endoscope in the statuses ST11 to ST12 is in the cleaned state, the hands of a staff member handling the endoscopes need to be clean. If the hands of the staff member handling the endoscope are dirty, the hands need to be washed. However, if the hands of the staff member handling the endoscope are clean, there is no need to wash the hands, and the work efficiency is high. Therefore, the staff identification unit 36 assigns the work of handling a cleaned endoscope to a staff member whose hands are in a clean state at that time.

As shown in FIG. 6, the staff management unit 34 manages the work status of staff members in association with the cleanliness of hands. That is, by managing the work status, the staff management unit 34 manages whether or not a staff member is in a state suitable for being assigned to the work of handling an endoscope in a cleaned state. More specifically, the staff management unit 34 manages whether or not the hands of the staff member are in a clean state. FIG. 6 shows the management conditions of the work statuses of staff members A to E at a given time, and it is identified that the hands of the staff members B and D are in a clean state at this time.

As an example, a case where the main cleaning in the cleaning device 16 is completed will be described. The cleaning device 16 transmits the completion information of the main cleaning to the management device 10 along with the device ID, and the acquirer 30 records the completion information of the main cleaning in the status information recording unit 44 in association with the scope ID recorded at the time of starting the main cleaning. At this time, the endoscope management unit 32 sets the status of the endoscope 3 to “main cleaning completed” (ST11).

In response to the transition of the status of an endoscope to ST11, the staff identification unit 36 searches for a staff member for transporting the endoscope on which the main cleaning has been performed to the storage 18 with reference to the state management table 64. The endoscope management unit 32 manages an endoscope in the status ST11 as an endoscope that is in a cleaned state. Therefore, the staff identification unit 36 searches for the existence of a staff member whose hands are clean and who is in an “available” state. With reference to FIG. 6, the staff identification unit 36 identifies that a staff member who meets the conditions is “staff member B”.

The notification processing unit 42 notifies the staff member B identified by the staff identification unit 36 of the details of the work to be performed for the endoscope. The details of the work may include information on the endoscope subjected to the work and the work type. When each staff member in the medical facility wears a headset, the notification processing unit 42 gives notification to the headset of the staff member B of voice data indicating the details of the work. The details of the work may be an instruction to take out the cleaned endoscope from the cleaning tank of the cleaning device 16 and store the endoscope in the storage 18. By receiving the voice instruction, the staff member B can promptly recognize the work to be performed.

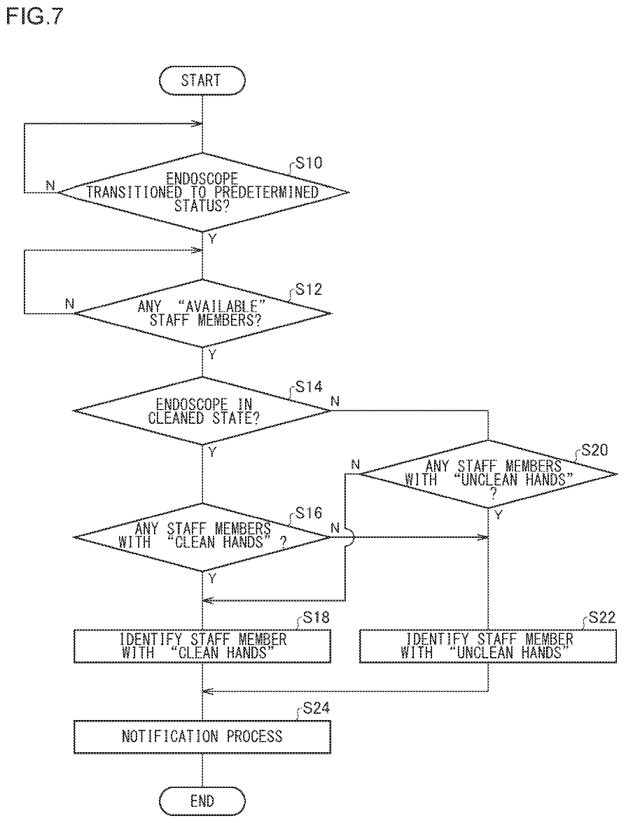

A staff identification process performed by the staff identification unit 36 will be described below. FIG. 7 shows a flowchart of the staff identification process. The endoscope management unit 32 monitors whether or not an endoscope 3 transitions to a predetermined status (N in S10). The predetermined status is a status that requires new work by a staff member and may be ST2, ST4, ST6, ST8, or ST11 as described above. The endoscope management unit 32 monitors the status of all endoscopes 3 used in the medical facility. Therefore, a flow shown in FIG. 7 is performed for each endoscope.

When the endoscope 3 transitions to the predetermined status (Y in S10), the endoscope management unit 32 requests the staff identification unit 36 to assign a staff member to the work related to the endoscope 3. The staff identification unit 36 acquires the state of each staff member by referring to the state management table 64 managed by the staff management unit 34.

The staff identification unit 36 searches for a staff member in the “available” state with reference to the state management table 64 shown in FIG. 6 (S12). The staff identification unit 36 determines that the staff member B and the staff member C are in the “available” state (Y in S12). If there is no staff member in the “available” state (N in S12), the staff identification unit 36 stays on stand-by until the state of a staff member becomes “available”.

The staff identification unit 36 determines whether or not the endoscope 3 that requires work is in a cleaned state (S14). When the endoscope 3 is in the status of ST2, ST4, ST6, or ST8, the staff identification unit 36 determines that the endoscope 3 is in an uncleaned state (N in S14). When the endoscope 3 is in the status of ST11, the staff identification unit 36 determines that the endoscope 3 is in a cleaned state (Y in S14).

When the endoscope 3 is in a cleaned state (Y in S14), the staff identification unit 36 checks whether there are any staff members who are in the “available” state and who do not need hand washing (S16). When there is an available staff member with “clean hands” (Y in S16), the staff identification unit 36 identifies the staff member (S18) and assigns work. In the state management table 64 shown in FIG. 6, since the available staff member with clean hands is the staff member B, the staff identification unit 36 identifies the staff member B and assigns the work related to the endoscope 3. If there is no available staff member with clean hands (N in S16), the staff identification unit 36 identifies an available staff member with “unclean hands” (S22) and assigns work. In this case, the staff member assigned to the work needs to carry out the work after washing their hands.

On the other hand, when the endoscope 3 is in an uncleaned state (N in S14), the staff identification unit 36 checks whether there are any staff members who are in the “available” state and who have unclean hands (S20). When there is an available staff member with “unclean hands” (Y in S20), the staff identification unit 36 identifies the staff member (S22) and assigns work. In the state management table 64 shown in FIG. 6, since the available staff member with unclean hands is the staff member C, the staff identification unit 36 identifies the staff member C and assigns the work related to the endoscope 3. If there is no available staff member with unclean hands (N in S20), the staff identification unit 36 identifies an available staff member with “clean hands” (S18) and assigns the work.

When the staff identification unit 36 identifies the staff member, the notification processing unit 42 notifies the staff member of the details of the work to be performed regarding the endoscope 3 (S24). The notification processing unit 42 may transmit voice data of the details of the work to the headset worn by the staff member. This allows the staff member to immediately grasp the details of the work and start the work.

In S18 and S22, there may be multiple staff members who meet the conditions. In this case, the staff identification unit 36 may identify all the staff members who meet the conditions, and the notification processing unit 42 may notify all the identified staff members of the details of the work to be performed.

When there are multiple staff members who meet the conditions, the staff identification unit 36 may narrow down the staff members to one staff member. For example, the staff identification unit 36 may identify one staff member according to a preset notification destination priority. Further, the staff identification unit 36 may identify one staff member assumed to be near a place where the assigned work is performed. For example, since it is assumed that a staff member who has just completed the same work as the work to be performed is near the work place, the staff identification unit 36 may identify the staff member. Further, the staff identification unit 36 may grasp the work amount of each staff member and conduct the identification of staff members for the purpose of equalizing the work amount.

The notification processing unit 42 may give notification to the headset of the staff member of voice data of the details of the work, and then give notification of voice data indicating each process constituting the work in the order of the processes. By listening to the procedure of each process through the headset, the staff member can accurately understand and carry out the work procedure. The voice data indicating each step of the work may be stored in the procedure storing unit 38. The notification processing unit 42 may read the voice data stored for the work assigned to the staff member from the procedure storing unit 38 and transmit the voice data to the headset of the staff member.

The determination unit 40 may have a function of determining whether or not the staff member has properly performed the work process. When the order in which IDs are to be read by an ID reading device and the timing of operating a predetermined button are determined as the work procedure, the determination unit 40 determines whether or not the work process is being properly performed based on information acquired by the acquirer 30. When the determination unit 40 determines that the work process is not being properly performed, the notification processing unit 42 may give notification to the headset of information indicating that the work process is not being properly performed.

Described above is an explanation on the present disclosure based on the embodiments. These embodiments are intended to be illustrative only, and it will be obvious to those skilled in the art that various modifications to constituting elements and processes could be developed and that such modifications are also within the scope of the present disclosure.

What is claimed is:

1. A medical care supporting system comprising:
a processor comprising hardware, wherein the processor is configured to:
manage whether or not an endoscope is in a cleaned state;
manage whether or not a medical staff member is in a state suitable for being assigned to work of handling an endoscope in a cleaned state;
identify a medical staff member who performs work related to the endoscope based on the state of the endoscope being managed and the state of the medical staff member being managed; and
notify the identified medical staff member of the details of the work to be performed for the endoscope.

2. The medical care supporting system according to claim 1, wherein
the processor is configured to manage whether or not the endoscope is in the cleaned state based on the work to be performed for the endoscope.

3. The medical care supporting system according to claim 1, wherein
the processor is configured to manage whether or not the medical staff member is in the state suitable for being assigned to the work of handling the endoscope in the cleaned state based on work that has been performed for the endoscope by the medical staff member.

4. The medical care supporting system according to claim 1, wherein
the processor is configured to give notification to a voice output device worn by the medical staff member of voice data indicating each process of the work.

5. A medical care supporting method comprising:
managing whether or not an endoscope is in a cleaned state; and
managing whether or not a medical staff member is in a state suitable for being assigned to work of handling an endoscope in a cleaned state, wherein
a management device identifies a medical staff member who performs work related to the endoscope based on the state of the endoscope being managed and the state of the medical staff member being managed; and
the management device notifies the identified medical staff member of the details of the work to be performed for the endoscope.

6. The medical care supporting method according to claim 5, wherein
managing the state of the endoscope includes managing whether or not the endoscope is in the cleaned state based on the work to be performed for the endoscope.

7. The medical care supporting method according to claim 5, wherein
managing the state of the medical staff member includes managing whether or not the medical staff member is in the state suitable for being assigned to the work of handling the endoscope in the cleaned state based on work that has been performed for the endoscope by the medical staff member.

8. The medical care supporting method according to claim 5, wherein
the management device gives notification to a voice output device worn by the medical staff member of voice data indicating each process of the work.
